(19) United States
(12) Patent Application Publication
(10) Pub. No.: US 2006/0100271 A1
(43) Pub. Date: May 11, 2006

(54) Stabilized aqueous ranitidine compositions

(76) Inventor: Keith Whitehead, Oldsmar, FL (US)

Correspondence Address: WYATT BARTON PRATT, 15 COLUMBIA AVENUE, HOPEWELL, NJ, 08525, US

(21) Appl. No.: 10/984,209

(22) Filed: Nov. 8, 2004

(51) Int. Cl. A61K 9/20; A61K 31/34

(52) U.S. Cl. 514/471; 424/464

(57) ABSTRACT

An ethanol free aqueous pharmaceutical ranitidine composition of ranitidine, or pharmaceutically acceptable salt thereof, is stabilized with an effective amount of hydroxyethyl cellulose. Preferred excipients include methyl paraben, and either sorbitol or sucrose.

### BACKGROUND OF THE INVENTION

1. Field of the Invention

The present invention pertains to a pharmaceutical liquid composition containing an active pharmaceutical ingredient of ranitidine stabilized with hydroxyethyl cellulose.

2. Brief Description of the Related Art

Ranitidine, [N-[2-[[[5-(dimethylamino)methyl-2-furanyl]methyl]thio]ethyl]-N′-methyl-2-nitro-1,1-ethenediamine], has been used as an active pharmaceutical ingredient for duodenal ulcers, particularly in its salt form of ranitidine hydrochloride. However, in liquid form, ranitidine has presented stability problems. U.S. Pat. No. 4,585,790, to Padfield et al., entitled Pharmaceutical Compositions discloses that the shelf life of aqueous based formulations containing ranitidine and/or one or more of its physiologically acceptable salts may be significantly enhanced if the pH of the formulation is adjusted within the range of 6.5-7.5. Additionally, U.S. Pat. No. 5,068,249, to Long, entitled Aqueous Rantidine Compositions Stabilized with Ethanol discloses that the stability of ranitidine in aqueous based formulations and more particularly aqueous based formulations for oral administration may be substantially enhanced by the addition of ethanol to the formulation. However, the use of ethanol to enhance the stability of ranitidine may be detrimental to patients using the drug, particularly children.

Accordingly, there is a need in the art to provide stable ranitidine compositions free of ethanol. The present invention addresses this and other needs.

### SUMMARY OF THE INVENTION

The present invention includes a stabilized aqueous pharmaceutical ranitidine composition, comprising ranitidine, or pharmaceutically acceptable salt thereof, stabilized with a stabilizing effective amount of hydroxyethyl cellulose, wherein the composition is essentially free of ethanol.

The present invention also includes a process for producing an aqueous pharmaceutical ranitidine composition comprising the steps of preparing a solution of ranitidine, or pharmaceutically acceptable salt thereof and mixing the ranitidine solution with an effectively stabilizing amount of hydroxyethyl cellulose, wherein the ranitidine-hydroxyethyl cellulose mixture is essentially free of ethanol.

Additionally, the present invention includes a pediatric formulation comprising an ethanol-free stabilized aqueous pharmaceutical of ranitidine.

Surprising it has been discovered that the stability of aqueous ranitidine formulations may be substantially enhanced by the addition of an effective amount of hydroxyethyl celllulose in the formulation.

Other features, advantages and embodiments of the invention will become apparent to those of ordinary skill in the art by the following description, accompanying examples and appended claims.

### DETAILED DESCRIPTION OF THE INVENTION

The present invention includes a stabilized aqueous pharmaceutical ranitidine composition comprising ranitidine, or pharmaceutically acceptable salt thereof, stabilized with an effective amount of hydroxyethyl cellulose, wherein the composition is essentially free of ethanol. The aqueous formulations of the present invention affords a syrup or other similarly created formulation for oral administration that is conveniently prepared by adding an aqueous solution of ranitidine, and/or one or more of its salts, together with hydroxyethyl cellulose, with other excipients preferably added. The aqueous formulations are preferably prepared using ranitidine in the form of its hydrochloride salt. The aqueous formulations herein provide novel, stable and pourable ranitidine pharmaceutical formulations, particularly in pediatric formulations.

In one preferred embodiment, the present invention includes an ethanol-free stabilized aqueous pharmaceutical ranitidine composition comprising ranitidine, or pharmaceutically acceptable salt thereof, an effective amount of hydroxyethyl cellulose to stabilize the ranitidine in solution, together with sorbitol. In another embodiment, the ranitidine is stabilized with the hydroxyethyl cellulose, and combined with sucrose. In these formulations, methyl paraben is preferably added. The oral solutions of the present invention include compositions free of glycerin, polyethlyene glycol or alcohols. These embodiments of the present invention are particularly applicable as a pediatric formulation to provide an ethanol-free stabilized aqueous pharmaceutical of ranitidine.

Compositions of the present invention are essentially or substantially free of ethanol when the amount of ethanol within the composition is sufficiently small as to not impar5t noticeable effect on the stability of the formulation and/or impart a physiological or pharmaceutical effect on a patient. Preferably, no ethanol is detectably present within the compositions of the present invention.

The oral solutions of the present invention include an appropriate amount of ranitidine, generally having an amount that is therapeutically effective in a convenient dosage unit for a given patient. Preferred amounts of ranitidine contained within the pharmaceutical formulation of the present invention include, for example without limitation, up to about 50 grams of ranitidine per 100 mL of oral solution, with preferred ranges of from about 0.5 grams to about 40 grams of ranitidine per 100 mL of oral solution, more preferably from about 1 gram to about 25 grams of ranitidine per 100 mL of oral solution, and most preferably from about 1.5 grams to about 10 grams of ranitidine per 100 mL of oral solution. As the relative amount of ranitidine is increased within a given volume of oral solution, such as over about 20 grams of rantidine per 100 mL, the oral solution becomes increasing problematic to readily taste-mask with the addition of sweeteners and flavoring agents. Preferably the ranitidine is in the form of a pharmaceutically acceptable salt, with the ranitidine more preferably in a pharmaceutically acceptable salt of ranitidine hydrochloride. Representative unit dosages of the present invention include ranitidine in an amount of from about 15 mg of ranitidine per 1 mL of oral solution to about 20 mg of ranitidine per 11 mL of oral solution, such as about 16.8 mg of ranitidine per 1 mL of oral solution.

Hydroxyethyl cellulose is a non-ionic water-soluble polymer derived from cellulose. It is a cellulose ether that is substantially unaffected by cations. It is believed that this provides a platform that is less affected by pH change and more tolerant to the presence of anions and organic co-solvents. Preferably, the hydroxyethyl cellulose is present in a ratio amount to the ranitidine of from about 1:16 to about 1:1 of hydroxyethyl cellulose to rantidine, more preferably from about 1:8 to about 1:2, still more preferably from about 1:6 to about 1:3, and most preferably of about 1:4.

When used herein, the terms active agent, active pharmaceutical ingredient, pharmaceutical actives, API, active compound, therapeutic agent, therapeutic ingredient, therapeutic compound and other like terms are used interchangeably and include salts and other pharmaceutical forms of the detailed compounds.

The aqueous formulation may include an oral formulation of ranitidine and/or one or more of its physiologically acceptable salts dissolved in water, an effectively stabilizing amount of hydroxyethyl cellulose and a preservative, with the pH of the aqueous formulation moderated by the use of appropriate buffer salts. Other conventional excipients such as a sweetener, a flavor and/or flavoring aids may be included. Sodium chloride is a preferred excipient for regulating the tonicity of the oral solution.

The present invention may include suitable buffers (also referred to herein as buffer salts or buffering agent). As used herein, the term “buffers” is intended to mean a compound used to resist a change in pH upon dilution or addition of acid or alkali. Preferred buffer salts include, by way of example and without limitation, potassium dihydrogen orthophosphate, disodium hydrogen orthophosphate, citric acid and disodium hydrogen orthophosphate, potassium phosphate dibasic, sodium phosphate dibasic, potassium metaphosphate, potassium phosphate, monobasic sodium acetate and sodium citrate anhydrous and dehydrate and other such like materials known in the art. Most preferably, sodium phosphate dibasic is used. Suitable buffers are generally selected to be chemically unreactive with the other ingredients that may be present in the oral solution, with the buffers present in amounts sufficient to provide the desired degree of pH buffering. Preferred pHs of the aqueous formulation of the present invention range from about 6.0 to about 8.0, such as for example from about 6.5 to about 7:5, particularly about 6.8 to about 7.4 and more particularly about 6.8 to about 7.1. Variations and adjustments of the pH of the aqueous formulations are preferably obtained by moderating the addition of the buffer salt(s).

Although hydroethyl cellulose provide a degree of viscosity control to the pharmaceutical formulation of the present invention, additional viscosity enhancing agents may be included in the compositions of the present invention, as appropriate, as determinable by one skilled in the art, to provide desired flow characteristic to the composition. The amount of viscosity enhancing agent in the formulation is preferably sufficient to give a solution with a viscosity in the range of 10 to 100 centipoises, with a more preferred viscosity range of from about 20 to 90 centipoises, a still more preferred viscosity range of from about 25 to 75 centipoises, and a most preferred viscosity range of from about 50 to 60 centipoises. Representative viscosity enhancing agents suitable for inclusion in the present invention include, for example without limitation, Xanthan gum, sorbitol, glycerol, sucrose or cellulose derivatives in addition to the hydroxyethyl cellulose such as carboxymethylcellulose or a salt thereof of a C₁₋₄ alkyl and/or a hydroxy-C₂₋₄ alkyl ether of cellulose, such as methylcellulose, ethylcellulose, hydroxypropylcellulose, hydroxyethylmethylcellulose and hydroxypropylmethylcellulose.

Representative preservatives suitable for use in the present invention include, for example without limitation, one or more alkyl hydroxybenzoates, such as methyl hydroxybenzoates, ethyl hydroxybenzoates, propyl hydroxybenzoates, butyl hydroxybenzoates and the like. Additional preservatives useful in the present invention include, but are not limited to, sodium benzoate, potassium sorbate, salts of edetate (also know as salts of ethylenediaminetetraacetic acid, or EDTA, such as disodium edetate) and antimicrobial agents including parabens (p-hydroxybenzoic acids esters) such as methyl paraben, ethylparaben, propylparaben, butylparaben and the like, and combinations thereof. The preservatives listed herein are exemplary, with the appropriate preservative and amount of a preservative incorporated into the composition as determinable by one skilled in the art for compatibility and efficacy of the preservative in a given oral solution. Techniques and methods for evaluating preservative efficacy in a given pharmaceutical formulations are readily known in the art. Parabens are preferred, with methyl paraben most preferred for use as preservative ingredients to add to the present pharmaceutical composition, although other pharmaceutically acceptable preservatives may be substituted therefor. Preservatives may be included in a given pharmaceutical formulation of the present invention as appropriate, with preferred amounts of up to 1 gram per 100 mL of the oral solution. More preferably the preservatives are included in amounts that range of from about 0.10 to about 0.75 grams per 100 mL of the oral solution, still more preferably from about 0.15 to about 0.5 grams per 100 mL of the oral solution, and most preferably from about 0.20 to about 0.4 grams per 100 mL of the oral solution. Methyl paraben is preferably present in amounts of from about 0.10% w/v to about 0.25% w/v, with preferred amounts of the methyl paraben ranging from about 0.15% w/v to about 0.18% w/v.

Coloring agents also may be incorporated in the oral solution of the present invention as determined by one skilled in the art to be appropriate, for chemical compatibility with other ingredients in the oral solution and the like. Coloring agents are generally used to provide an appealing color to the oral solution. Suitable coloring agents for use in pharmaceutical oral solutions are well known in the art. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and iron oxide (black, red, yellow), other F.D. & C. dyes and natural coloring agents such as grape skin extract, beet red powder, beta-carotene, annato, carmine, turmeric, paprika, combinations thereofand other such materials known to those skilled in the art.

The pharmaceutical formulation of the present invention preferably contains flavoring agents (herein referred to also as flavorants), sweetening agents, and combinations thereof to mask the inherently bitter taste associated with ranitidine, and thereby improving the palatability of the oral solution of the present invention. Flavorants are used to impart a pleasant flavor and often odor to a pharmaceutical preparation. Suitable flavoring agents include natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, strawberry flavor, lemon flavor, grapefruit flavor and “mint” flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the oral solution in amounts effective to provide a palatable flavor to the oral solution. The amount of flavoring agent may depend on a number of factors, including the desired organoleptic effect. The precise amount of sweetening and/or flavoring agent(s) depends on the properties of the agent(s) used, however generally in an amount that is sufficient to mask the bitter taste associated with ranitidine as determinable by one skilled in the art. However, flavoring agents are generally present in the oral solution in amounts in the range of from greater than about 0 grams to about 10 grams per 100 mL of the oral solution, with preferred amounts of from about 2 grams to about 5 grams per 100 mL. Sweeteners suitable for inclusion in the present invention may be determined by one skilled in the art including, for example without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfam K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combination thereof. Presently preferred as a sugar sweetener is sorbitol. The amount of sugar sweetener used in the oral solution varies with the degree of sweetening desired for the particular formulation as determinable by one skilled in the art, with preferred amounts of sugar sweetener ranging from greater than about 0 grams to about 100 grams sugar sweetener per 100 mL of the oral solution, more preferably from about 20 grams to about 95 grams per 100 mL of oral solution, still more preferably from 30 grams to about 90 grams sugar sweetener per 100 mL of the oral solution, and most preferably from about 40 grams to about 85 grams per 100 mL of oral solution. Sugar sweeteners may be replaced or augmented by water soluble artificial sweeteners, such as the suitable artificial sweeteners previously listed and mixtures thereof. The amount of artificial sweetener used in the oral solution may vary to provide an appropriate amount of sweetness to the oral solution as determinable by one skilled in the art, generally in amounts similar to those of sugar sweeteners described above. Mixtures of sweetening and/or flavoring agents are preferably used.

The aqueous pharmaceutical ranitidine composition of the present invention is produced through a process that includes the steps of preparing a solution of ranitidine, or pharmaceutically acceptable salt thereof, and mixing the ranitidine solution with an effectively stabilizing amount of hydroxyethyl cellulose, while maintaining the ranitidine-hydroxyethyl cellulose mixture essentially free of ethanol. Preparation of the aqueous pharmaceutical composition of the present invention is accomplished using ingredients of a purity such that it is suitable for administration to patients. Generally, the pharmaceutical formulation contains at least one conventional pharmaceutical excipient in addition to the hydroxyethyl cellulose and ranitidine and/or physiologically acceptable salts thereof.

The present invention provides methods of treating a subject (e.g., mammal, particularly humans) comprising administering to a subject in need of such treatment a therapeutically effective amount of at least one active ingredient, formulation thereof, or unit dose forms thereof, each as described herein. As used herein, the term “treatment”, or a derivative thereof, contemplates partial or complete inhibition of the stated disease state such as, for example, active duodenal ulcers, when an active ingredient of the present invention is administered prophylactically or following the onset of the disease state for which such active ingredient of the present invention is administered. For the purposes of the present invention, “prophylaxis” refers to administration of the active ingredient(s) to a mammal to protect the mammal from any of the disorders set forth herein, as well as others. Other examples of such conditions that may be treated include maintenance of healing of gastric ulcers and maintenance of healing of erosive esophagitis.

The typical active daily dose of the ranitidine depends on various factors such as, for example, the individual requirement of each patient, the route of administration, and the disease. An attending physician may adjust the dosage rate based on these and other criteria if he or she so desires. As an example, a suitable oral dosage form may encompass from about 37.5 to about 150 mg total daily dose, typically administered in one single dose or equally divided doses. A more preferred range is from about 75 mg to about 150 mg total daily dose, and a most preferred range is from about 75 mg to about 100 mg total daily dose. It should be appreciated that daily doses other than those described above may be administered to a subject, as appreciated by an attending physician. The stabilized aqueous pharmaceutical ranitidine composition of the present invention preferably is administered as a pediatric formulation in appropriate unit dosage form.

The ranitidine API may be used as a single active agent, or may be combined with other active agents, vitamins, minerals, dietary supplements, etc. The therapeutic compound(s) contained within the present device can be formulated as its pharmaceutically acceptable salts. As used herein, “pharmaceutically acceptable salts” refers to derivatives of the disclosed compounds wherein the therapeutic compound is modified by reacting it with an acid or base as needed to form an ionically bound pair. Examples of pharmaceutically acceptable salts include conventional non-toxic salts or the quaternary ammonium salts of the parent compound formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. The pharmaceutically acceptable salts of the present invention can be synthesized from the parent therapeutic compound which contains a basic or acidic moiety by conventional chemical methods. Lists of other suitable salts are found in Remington's Pharmaceutical Sciences, 17^{th} ed., Mack Publishing Company, Easton, Pa., 1985, p. 1418, the relevant disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The amount of therapeutic compound incorporated in each device of the invention includes at least one or more dosage form and can be selected according to known principles of pharmacy. An effective amount of therapeutic compound is specifically contemplated. By the term “effective amount”, it is understood that, with respect to, for example, pharmaceuticals, a pharmaceutically effective amount is contemplated. A pharmaceutically effective amount is the amount or quantity of a drug or pharmaceutically active substance which is enough for the required or desired therapeutic response, or in other words, the amount, which is sufficient to elicit an appreciable biological response when, administered to a patient. The appreciable biological response may occur as a result of administration of single or multiple unit doses of an active substance. Depending upon the active substance used and upon the amount of active substance present in a particular device according to the invention, a unit dose may comprise one or more such devices.

Formulations of an ethanol-free stabilized aqueous pharmaceutical ranitidine composition of the present invention are illustrated in the examples below. Comparative examples that include ethanol and/or hydroxypropyl methyl cellulose components are listed below as Comparative Examples. As used herein, w/v represents weight per volume measurement and v/v represents volume per volume measurement.

### EXAMPLE 1

An ethanol-free stabilized aqueous pharmaceutical ranitidine composition of the present invention was formulated by mixing a solution of the ranitidine hydrochloride together with the excipients, except hydroxyethyl cellulose, in purified water was added with mixing to a dispersion of the hydroxyethyl cellulose in purified water. The formulation included Ranitidine HCl (16.8 gm/mL), hydroxyethyl cellulose (0.45% w/v), sodium saccharin (0.15% w/v), sodium chloride (0.05% w/v), sodium phosphate dibasic (0.761% w/v), propylene glycol (2% v/v), sorbitol (10% v/v), propylparaben (0.02% w/v), butylparaben (0.02% w/v), spearmint flavor (0.5% v/v) and water (Q.S. 1000 mL). The composition showed a slight color change with the parabens remaining in solution.

### EXAMPLE 2

An ethanol-free stabilized aqueous pharmaceutical ranitidine composition of the present invention was formulated by mixing a solution of the ranitidine hydrochloride together with the excipients, except hydroxyethyl cellulose, in purified water was added with mixing to a dispersion of the hydroxyethyl cellulose in purified water. The formulation included Ranitidine HCl (16.8 gm/mL), hydroxyethyl cellulose (0.25% w/v), sodium saccharin (0.15% w/v), sodium chloride (0.05% w/v), sodium phosphate dibasic (0.761% w/v), propylene glycol (2% v/v), sorbitol (10% v/v), propylparaben (0.02% w/v), butylparaben (0.02% w/v), spearmint flavor (0.5% v/v) and water (Q.S. 1000 mL). The composition showed a slight color change with the parabens remaining in solution.

### EXAMPLE 3

### Ethanol, Glycerin & Sorbitol Free

A Ranitidine Syrup, having a concentration of 15 mg/mL, was formulated with sodium chloride 0.05% w/v (weight per volume), hydroxyethyl cellulose 0.35% w/v, sodium saccharin 0.15% w/v, sodium phosphate dibasic 0.35% w/v, sugar(sucrose, NF) 50.0% w/v, Ranitidine Hydrochloride (each 1 mL contains 16.8 mg of Ranitidine-hydrochloride equivalent to 15 mg of Ranitidine), methylparaben 0.18% w/v, N&A Spearmint Flavor (614036) 0.5% v/v (volume per volume) and purified water (approximately 55% v/v). The composition showed a slight color change with the paraben remaining in solution.

### EXAMPLE 4

### Ethanol, Glycerin & Sugar Free

A Ranitidine Syrup, having a concentration of 15 mg/mL, was formulated with sodium chloride 0.05% w/v (weight per volume), hydroxyethyl cellulose 0.35% w/v, sodium saccharin 0.15% w/v, sodium phosphate dibasic 0.35% w/v, sorbitol 10.0% v/v, Ranitidine Hydrochloride (each 1 mL contains 16.8 mg of Ranitidine-hydrochloride equivalent to 15 mg of Ranitidine), methylparaben 0.15% w/v, N&A Spearmint Flavor (614036) 0.5% v/v (volume per volume) and purified water (approximately 55% v/v). The composition showed a slight color change with the parabens remaining in solution.

### COMPARATIVE EXAMPLE 1

### Using Hydroxypropyl Methyl Cellulose in place of Hydroxyethyl Cellulose

An aqueous ranitidine composition was formulated by mixing a solution of ranitidine hydrochloride together with the excipients, except hydroxypropyl methyl cellulose, in purified water was added with mixing to a dispersion of the hydroxypropyl methyl cellulose in purified water. The formulation included Ranitidine HCl (16.8 gm/mL), hydroxypropyl methyl cellulose (0.4% w/v), sodium saccharin (0.15% w/v), sodium chloride (0.05% w/v), sodium phosphate dibasic (0.761% w/v), propylene glycol (2% v/v), sorbitol (10% v/v), propylparaben (0.04% w/v), butylparaben (0.04% w/v), spearmint flavor (0.5% v/v) and water (Q.S. 1000 mL). The oral solution experienced a dark red color change evidencing degradation, fisheye formations within the oral solution and difficulty of the parabens to go into solution.

### COMPARATIVE EXAMPLE 2

### Using Hydroxypropyl Methyl Cellulose in Place of Hydroxyethyl Cellulose

An aqueous ranitidine composition was formulated by mixing a solution of ranitidine hydrochloride together with the excipients, except hydroxypropyl methyl cellulose, in purified water was added with mixing to a dispersion of the hydroxypropyl methyl cellulose in purified water. The formulation included Ranitidine HCl (16.8 gm/mL), hydroxypropyl methyl cellulose (0.4% w/v), sodium saccharin (0.15% w/v), sodium chloride (0.05% w/v), sodium phosphate dibasic (0.761% w/v), propylene glycol (2% v/v), sorbitol (10% v/v), propylparaben (0.02% w/v), butylparaben (0.02% w/v), spearmint flavor (0.5% v/v) and water (Q.S. 1000 mL). The oral solution experienced a dark red color change evidencing degradation, fisheye formations within the oral solution and difficulty of the parabens to go into solution.

### COMPARATIVE EXAMPLE 3

### Using Hydroxypropyl Methyl Cellulose in Place of Hydroxyethyl Cellulose

An aqueous ranitidine composition was formulated by mixing a solution of ranitidine hydrochloride together with the excipients, except hydroxypropyl methyl cellulose, in purified water was added with mixing to a dispersion of the hydroxypropyl methyl cellulose in purified water. The formulation included Ranitidine HCl (16.8 gm/mL), hydroxypropyl methyl cellulose (0.4% w/v), sodium saccharin (0.15% w/v), sodium chloride (0.05% w/v), sodium phosphate dibasic (0.761% w/v), propylene glycol (2% v/v), sorbitol (10% v/v), propylparaben (0.05% w/v), butylparaben (0.05% w/v), spearmint flavor (0.5% v/v) and water (Q.S. 1000 mL). The oral solution experienced a dark red color change evidencing degradation, fisheye formations within the oral solution and difficulty of the parabens to go into solution.

### COMPARATIVE EXAMPLE 4

### Using Hydroxypropyl Methyl Cellulose and Ethanol

An aqueous ranitidine composition was formulated by mixing a solution of ranitidine hydrochloride together with ethanol and other excipients, except hydroxypropyl methyl cellulose, in purified water was added with mixing to a dispersion of the hydroxypropyl methyl cellulose in purified water. The formulation included Ranitidine HCl (16.8 gm/mL), hydroxypropyl methyl cellulose (0.4% w/v), sodium saccharin (0.15% w/v), sodium chloride (0.05% w/v), sodium phosphate dibasic (0.761% w/v), propylene glycol (2% v/v), sorbitol (10% v/v), propylparaben (0.05% w/v), butylparaben (0.05% w/v), spearmint flavor (0.5% v/v) and water (Q.S. 1000 mL). The oral solution experienced a dark red color change evidencing degradation and difficulty of the parabens to go into solution.

The foregoing summary, description, and examples of the invention are not intended to be limiting, but are only exemplary of the inventive features which are defined in the claims.

What is claimed is:

1. A stabilized aqueous pharmaceutical ranitidine composition, comprising ranitidine, or pharmaceutically acceptable salt thereof, stabilized with a stabilizing effective amount of hydroxyethyl cellulose, wherein the composition is essentially free of ethanol.

2. The composition of claim 1, wherein the hydroxyethyl cellulose is present in a ratio amount to the ranitidine of from about 1:16 to about 1:1 of hydroxyethyl cellulose to rantidine.

3. The composition of claim 2, wherein the hydroxyethyl cellulose is present in a ratio amount to the ranitidine of from about 1:8 to about 1:2 of hydroxyethyl cellulose to rantidine.

4. The composition of claim 3, wherein the hydroxyethyl cellulose is present in a ratio amount to the ranitidine of from about 1:6 to about 1:3 of hydroxyethyl cellulose to rantidine.

5. The composition of claim 5, wherein the hydroxyethyl cellulose is present in a ratio amount to the ranitidine of about 1:4 hydroxyethyl cellulose to rantidine.

6. The composition of claim 1, further comprising sorbitol.

7. The composition of claim 6, wherein the sorbitol is present in an amount of from about 5% v/v to about 15% v/v.

8. The composition of claim 7, wherein the sorbitol is present in an amount of about 10% v/v.

9. The composition of claim 1, further comprising sucrose.

10. The composition of claim 9, wherein the sucrose is present in an amount of from about 25% w/v to about 75% w/v.

11. The composition of claim 10, wherein the sucrose is present in an amount of about 50% w/v.

12. The composition of claim 11, wherein the one or more preservatives comprises one or more paraben compounds.

13. The composition of claim 12, wherein the one or more paraben compounds is methyl paraben.

14. The composition of claim 13, wherein the methyl paraben is present in an amount of from about 0.10% w/v to about 0.25% w/v.

15. The composition of claim 14, wherein the methyl paraben is present in an amount of from about 0.15% w/v to about 0.18% w/v.

16. The composition of claim 1, wherein the pharmaceutically acceptable salt of ranitidine is ranitidine hydrochloride.

17. A unit dosage of the composition of claim 1, wherein the ranitidine hydrochloride is present in an amount of from about 15 mg of ranitidine per 1 mL of oral solution to about 20 mg of ranitidine per 1 mL of oral solution.

18. A unit dosage of the composition of claim 17, wherein the ranitidine hydrochloride is present in an amount of about 16.8 mg of ranitidine per 1 mL of oral solution.

19. A process for producing an aqueous pharmaceutical ranitidine composition, comprising the steps of:
preparing a solution of ranitidine, or pharmaceutically acceptable salt thereof; and,
mixing the ranitidine solution with an effectively stabilizing amount of hydroxyethyl cellulose, wherein the ranitidine-hydroxyethyl cellulose mixture is essentially free of ethanol.

20. A pediatric formulation comprising the ethanol-free stabilized aqueous pharmaceutical ranitidine composition of claim 1.
